(19) United States
(12) Patent Application Publication
(10) Pub. No.: US 2003/0139606 A1
(43) Pub. Date: Jul. 24, 2003

(54) Process for preparing 5-methylisoxazole-4-carboxylic- (4'-trifluoromethyl)-anilide

(76) Inventors: Anup Kumar Ray, Staten Island, NY (US); Hiren Kumar V. Patel, Fords, NJ (US); Mahendra R. Patel, East Brunswick, NJ (US)

Correspondence Address: THOMAS HOXIE, NOVARTIS, PATENT AND TRADEMARK DEPARTMENT, ONE HEALTH PLAZA 430/2, EAST HANOVER, NJ, 07936-1080, US

(21) Appl. No.: 10/287,936

(22) Filed: Nov. 5, 2002

Related U.S. Application Data
(60) Provisional application No. 60/344,951, filed on Nov. 9, 2001.

(51) Int. Cl. C07D 261/18

(52) U.S. Cl. 548/248

(57) ABSTRACT

A process for preparing 5-methylisoxazole-4-carboxylic-(4′-trifluoromethyl)-anilide comprising: (a) reacting ethylacetoacetate, triethylorthoformate, and acetic anhydride at a temperature of from about 75° C. to about 150° C., to form ethyl ethoxymethyleneacetoacetic ester; (b) combining the ethyl ethoxymethyleneacetoacetic ester with sodium acetate or a salt of trifluoroacetic acid in the presence of hydroxylamine sulfate at a temperature of from about −20° C. to 10° C., to form ethyl-5-methylisoxazole-4-carboxylate; (c) reacting the ethyl-5-methylisoxazole-4-carboxylate with a strong acid to form 5-methylisoxazole-4-carboxylic acid; (d) reacting the crystallized 5-methylisoxazole-4-carboxylic acid with thionyl chloride to form 5-methylisoxazole-4-carbonyl chloride; and (e) reacting the 5-methylisoxazole-4-carbonyl chloride with trifluoromethyl aniline and an amine base at a temperature of from about 0° C. to about 50° C. to form 5-methylisoxazole-4-carboxylic-(4′-trifluoromethyl)-anilide. The process of the invention is especially advantageous for preparing 5-methylisoxazole-4-carboxylic-(4′-trifluoromethyl)-anilide, since the process: (1) eliminates or reduces the formation of the by-product 2-cyanoacetoacetic-1-(4′-trifluoromethyl)-anilide (CATA), generally as low as 0.0006%; (2) eliminates or reduces the formation of isomeric impurity ethyl-3-methyisoxazole-4-carboxylate and its corresponding acid as low as 0.1%, (3) produces a high quality of 5-methylisoxazole-4-carboxylic-(4′-trifluoromethyl)-anilide generally having 99.8-100% HPLC potency; and (4) does not require distillation of the isoxazole ester.

### FIELD OF THE INVENTION

The present invention provides an improved process for preparing 5-methylisoxazole-4-carboxylic-(4′-trifluoromethyl)-anilide or Leflunomide.

### BACKGROUND OF THE INVENTION

U.S. Pat. No. 4,284,786 (the '786 process) describes a process for preparing 5-methylisoxazole-4-carboxylic-(4′-trifluoromethyl)-anilide. The '786 process involves a direct condensation of 4-trifluoromethylaniline with diketene, resulting in an acetoacetic acid derivative which upon reaction with orthoformic acid ester yields 2-alkoxymethyleneacetoacetic acid anilide. The 2-alkoxymethyleneacetoacetic acid anilide is treated with hydroxylamine hydrochloride in the presence of strong base sodium hydroxide or sodium carbonate to yield 5-methylisoxazole-4-carboxylic-(4′-trifluoromethyl)-anilide.

The disadvantages of the '786 process are that it (i) requires very costly 4-trifluoromethylaniline to be used in the first step; (ii) subsequent steps show a diminishing trend in the yield of 5-methylisoxazole-4-carboxylic-(4′-trifluoromethyl)-anilide; and (iii) during cyclization of the isoxazole ring formation with the attached anilide linkage in the basic medium under refluxing condition, there is hydrolysis of amide bond under basic conditions resulting in the formation of degraded products 5-methylisoxazole-4-carboxylic acid and 4-trifluoromethylaniline along with the formation of by-product by the elimination of highly labile hydrogen from 3-position of the isoxazole ring.

German Patent No. 634,286 (the '286 process) describes a process for preparing 5-methylisoxazole-4-carboxylic-(4-trifluoromethyl)-anilide from acid chloride of 5-methyl-isoxazole-4-carboxylic acid. The '286 process uses thionyl chloride as a chlorinating agent to generate acid chloride.

A disadvantage of the '286 process is that it uses 5-methylisoxazole-4-carboxylic acid chloride in contact with strong base potassium hydroxide or dropwise addition of acid chloride to the basic environment of 4-trifluoromethylaniline leads to an undesirable side reaction to generate the acid as well as a by-product, 2-cyanoacetoacetic-1-(4′-trifluoromethyl)-anilide (CATA). The CATA is a by-product of 5-methylisoxazole-4-carboxylic-(4′-trifluoromethyl)-anilide, and thus, is very difficult to get rid of even by repeated crystallization.

WO 01/60363 describes a process for preparing 5-methylisoxazole-4-carboxylic-(4-trifluoromethyl)-anilide using 5-methylisoxazole-4-carboxylic acid to prepare acid chloride, which is reacted with 4-trifluoromethylaniline in the presence of strong base. The process described in WO 01/60363 is similar to the '286 process except that in the last step a biphasic solution of organic solvent and water (same as '786 process) under heating condition is utilized, instead of one solvent system, as described in the '286 process. In hot condition under aqueous and basic environment, there is always a possibility of hydrolysis of acid chloride and amide bond as well as the formation of the by-product CATA by abstraction of the base sensitive proton at 3-position of the isoxazole ring.

Thus, it would be desirable to develop a process for preparing pharmaceutically acceptable 5-methylisoxazole-4-carboxylic-(4′-trifluoromethyl)-anilide with high quality API without forming by-products such as CATA and isomeric impurities that are present in the prior art processes.

### SUMMARY OF THE INVENTION

The invention provides a process for preparing 5-methylisoxazole-4-carboxylic-(4′-trifluoromethyl)-anilide comprising: (a) reacting ethylacetoacetate, triethylorthoformate, and acetic anhydride at a temperature of from about 75° C. to about 150° C., to form ethyl ethoxymethyleneacetoacetic ester; (b) combining the ethyl ethoxymethyleneacetoacetic ester with sodium acetate or a salt of trifluoroacetic acid in the presence of hydroxylamine sulfate at a temperature of from about −20° C. to 10° C., to form ethyl-5-methylisoxazole-4-carboxylate; carboxylate; (c) reacting the ethyl-5-methylisoxazole-4-carboxylate with a strong acid to form 5-methylisoxazole-4-carboxylic acid; (d) reacting the crystallized 5-methylisoxazole-4-carboxylic acid with thionyl chloride to form 5-methylisoxazole-4-carbonyl chloride; and (e) reacting the 5-methylisoxazole-4-carbonyl chloride with trifluoromethyl aniline and an amine base at a temperature of from about 0° C. to about 50° C. to form 5-methylisoxazole-4-carboxylic-(4′-trifluoromethyl)-anilide.

According to another aspect, the invention provides a process for preparing 5-methylisoxazole-4-carboxylic-(4′-trifluoromethyl)-anilide comprising: (a) reacting ethylacetoacetate, triethylorthoformate, and acetic anhydride at a temperature of from about 75° C. to about 150° C., to form a mixture comprising ethyl ethoxymethyleneacetoacetic ester and a non-reactive component; (a′) separating the non-reactive component from the mixture formed in Step (a) to yield ethyl ethoxymethyleneacetoacetic ester; (b) combining the ethyl ethoxymethyleneacetoacetic ester formed in Step (a′) with sodium acetate or a salt of trifluoroacetic acid in the presence of hydroxylamine sulfate at a temperature of from about −20° C. to 10° C., to form crude ethyl-5-methylisoxazole-4-carboxylate; (b′) purifying the crude ethyl-5-methylisoxazole-4-carboxylate to form ethyl-5-methylisoxazole-4-carboxylate; (c) reacting the ethyl-5-methylisoxazole-4-carboxylate formed in Step (b′) with a strong acid to form 5-methylisoxazole-4-carboxylic acid; (c′) crystallizing the 5-methylisoxazole-4-carboxylic acid to form crystallized 5-methylisoxazole-4-carboxylic acid; (d) reacting the crystallized 5-methylisoxazole-4-carboxylic acid with thionyl chloride to form 5-methylisoxazole-4-carbonyl chloride; and (e) reacting the 5-methylisoxazole-4-carbonyl chloride with trifluoromethyl aniline and an amine base at a temperature of from about 0° C. to about 50° C. to form 5-methylisoxazole-4-carboxylic-(4′-trifluoromethyl)-anilide.

In the process of the invention, a reverse addition technique is employed which reduces the possibility of forming CATA, which is a by-product. In the reverse addition technique, trifluoromethyl aniline is mixed with triethylamine and added dropwise to the acid chloride of 5-methylisoxazole-4-carboxylic acid at low temperature with proper stirring. A slow addition of trifluoromethyl aniline (TFMA) and triethylamine by vigorous stirring provides immediate dispersion, thus limiting the localized basified zone, which generates the by-product CATA. The present inventors have determined that by using hydroxylamine sulfate instead of hydroxylamine hydrochloride, a much clear reaction mixture with drastic reduction of isomeric impurities is achieved, as compared to prior art processes.

The process of the invention is especially advantageous for preparing 5-methylisoxazole-4-carboxylic-(4′-trifluoromethyl)-anilide, since the process: (1) eliminates or reduces the formation of the by-product CATA generally as low as 0.0006%; (2) eliminates or reduces the formation of isomeric impurity ethyl-3-methyisoxazole-4-carboxylate and its corresponding acid as low as 0.1%, (3) produces a high quality of 5-methylisoxazole-4-carboxylic-(4′-trifluoromethyl)-anilide generally having 99.8-100% HPLC potency; and (4) does not require distillation of the isoxazole ester.

### DESCRIPTION OF THE INVENTION

The process of the invention is used to prepare 5-methylisoxazole-4-carboxylic-(4′-trifluoromethyl)-anilide or Leflunomide. The process involves Steps (a) through (e). In Step (a) ethylacetoacetate, triethylorthoformate, and acetic anhydride are reacted at a temperature of from about 75° C. to about 150° C., to form a mixture comprising ethyl ethoxymethyleneacetoacetic ester and a non-reactive component. The temperature in Step (a) is preferably from 90° C. to 120° C., more preferably from 100° C. to 110° C. The non-reactive component may optionally be separated from the mixture formed in Step (a) to yield the ethyl ethoxymethyleneacetoacetic ester. A preferred method of separation is distillation under reduced pressure.

In Step (b), the ethyl ethoxymethyleneacetoacetic ester formed in Step (a) is reacted with hydroxylamine sulfate in the presence of sodium acetate and/or a salt of trifluoroacetic acid at a temperature of from about −20° C. to about 10° C., to form crude ethyl-5-methylisoxazole-4-carboxylate. The temperature in Step (b) is preferably −20° C. to 0° C., more preferably from −10° C. to 0° C., and most preferably about −5° C. The salt of trifluoroacetic acid is preferably a sodium salt.

A preferred method to achieve the low temperature in Step (b) is by using a salt-ice-acetone bath enveloping the reactor. While not wishing to be bound by any particular theory, the present inventors believe that the low temperature employed in Step (b) increases the regioselectivity of the attack by nitrogen lone pair of hydroxylamine sulfate towards the ethoxymethylene carbon instead of carbonyl carbon thus limiting the formation of the isomeric impurity, ethyl-3-methylisoxazole-4-carboxylate and its corresponding acid in the subsequent step. Optionally, the crude ethyl-5-methylisoxazole-4-carboxylate may be purified to form ethyl-5-methylisoxazole-4-carboxylate. Methods of purification are known to those skilled in the art.

Step (b) may optionally be conducted in the presence of a solvent. Suitable solvents include alcohols, such as ethanol and isopropanol.

Preferably, in Step (b), a reverse addition technique is employed wherein the hydroxyl amine sulfate solution is added drop-wise to the reaction mixture at the above temperature, thus allowing control over localized concentration of the nucleophile, nitrogen lone pair of hydroxyl amine, and providing regioselectivity towards the ethoxymethylene carbon.

Sodium acetate and a salt of trifluoroacetic acid are weak bases. It is noted that the '286 process and the '786 process employ a strong alkali such as sodium hydroxide or sodium carbonate to form the ethyl-5-methylisoxazole-4-carboxylate. The present inventors have determined that the presence of a strong alkali used to prepare the ethyl-5-methylisoxazole-4-carboxylate results in a significantly higher amount of isomeric impurity and by-products.

IIn one embodiment of the invention, the crude ethyl-5-methylisoxazole-4-carboxylate ester formed in Step (b) is used in Step (c) without a distillation or purification step.

In Step (c), the ethyl-5-methylisoxazole-4-carboxylate formed in Step (b) is reacted with a strong acid to form 5-methylisoxazole-4-carboxylic acid. Any strong acid may be used provided that it is capable of hydrolyzing the carboxylate group of the ethyl-5-methylisoxazole-4-carboxylate. Examples of strong acids include sulfuric acid, hydrochloric acid, and phosphoric acid.

Optionally, Step (c′) is employed in the process of the invention, wherein the 5-methylisoxazole-4-carboxylic acid formed in Step (c) is crystallized to form crystallized 5-methylisoxazole-4-carboxylic acid. A solvent is preferably used in the crystallization. A preferred method of crystallizing involves combining the 5-methylisoxazole-4-carboxylic acid with a solvent and heating the mixture for a sufficient time and at a sufficient temperature to crystallize the 5-methylisoxazole-4-carboxylic acid. Preferred solvents for use in Step (c′) are selected from toluene, acetic acid, ethyl acetate, acetonitrile, 1,2-dichloroethane, 1,1-diethoxypropane, 1,1-diethoxymethane, isopropyl ether, dimethyl acetamide, and chlorinated solvents such as chloroform, methylene chloride, ethylene chloride, carbon tetrachloride and chlorobenzene. A combination of solvents may also be used. More preferably, the solvent in Step (c′) is a toluene and acetic acid mixture.

In Step (d), the crystallized 5-methylisoxazole-4-carboxylic acid formed in Step (c′) is reacted with thionyl chloride to form 5-methylisoxazole-4-carbonyl chloride. Preferably, the thionyl chloride is free from water. A solvent is optional in Step (d). It is within the scope of the invention that an excess of thionyl chloride is used wherein the thionyl chloride functions as a reactant and solvent. Preferred solvents for use in Step (d) include toluene, ethyl acetate, acetonitrile, 1,2-dichloroethane, dimethyl acetamide, and chlorinated solvents such as chloroform, methylene chloride, ethylene chloride, carbon tetrachloride and chlorobenzene. A combination of solvents may also be used. More preferably, the solvent in Step (d) is toluene.

In Step (e), the 5-methylisoxazole-4-carbonyl chloride formed in Step (d) is reacted with trifluoromethyl aniline (TFMA) and an amine base at a temperature of from about 0° C. to about 50° C. to form 5-methylisoxazole-4-carboxylic-(4′-trifluoromethyl)-anilide. The temperature in Step (e) is preferably 0° C. to 20° C., and more preferably from 5° C. to 15° C. Preferred amine bases are selected from triethylamine; N,N-diisopropylethylamine; and N,N′-diisopropylethylenediamine. A combination of amine bases may also be used. More preferably, the amine base is triethylamine.

The 5-methylisoxazole-4-carboxylic-(4′-trifluoromethyl)-anilide product may be isolated or purified. Preferably, the 5-methylisoxazole-4-carboxylic-(4′-trifluoromethyl)-anilide formed in Step (e) is crystallized. A preferred method of crystallizing involves combining the 5-methylisoxazole-4-carboxylic-(4′-trifluoromethyl)-anilide with a solvent and heating the mixture for a sufficient time and at a sufficient temperature to crystallize the 5-methylisoxazole-4-carboxylic-(4′-trifluoromethyl)-anilide.

The 5-methylisoxazole-4-carboxylic-(4′-trifluoromethyl)-anilide or Leflunomide product, which is prepared by the process of the invention, is useful as an anti-inflammatory, analgesic, or anti-pyretic. The 5-methylisoxazole-4-carboxylic-(4′-trifluoromethyl)-anilide is especially useful for treating rheumatoid arthritis.

The following non-limiting examples illustrate further aspects of the invention.

### EXAMPLE 1

### Comparison

Preparation of Leflunomide Using the '286 Process

Ethylacetoacetate was reacted with triethylorthoformate to form ethyl ethoxymethyleneacetoacetic ester. Cyclization of the ethyl ethoxymethyleneacetoacetic ester was performed with hydroxylamine hydrochloride in aqueous medium in the presence of potassium carbonate or sodium carbonate or alkali metal hydroxide. The product, ethyl-5-methylisoxazole-4-carboxylate, was hydrolyzed with a mixture of acetic acid and concentrated hydrochloric acid (2:1) to yield 5-methylisoxazole-4-carboxylic acid. This carboxylic acid was converted to the carboxylic acid halide. The halide when reacted with 4-trifluoroaniline to yield 5-methylisoxazole-4-carboxylic-(4′-trifluoromethyl)-anilide.

The process in the final step yielded CATA, which is a by-product generated at the level of 6-8%. CATA is generated under basic and refluxing condition. Isomeric impurity, ethyl-3-methylisoxazole-4-carboxylate was generated through the non-specific attack by nitrogen lone pair to carbonyl carbon of ethyl ethoxymethyleneacetoacetate during the synthesis of the intermediate ethyl-5-methylisoxazole-4-carboxylate at a level of 10.4%. FIG. 1 illustrates the formation of the isomeric impurity during the synthesis of the drug substance intermediate (ethyl-5-methylisoxazole-4-carboxylate):

[image]

In FIG. 1, the isomer (II) co-elutes with the drug substance intermediate (I) in reverse phase HPLC. This intermediate (II) is carried forward in the form of constitutional isomeric impurity to the final API. This isomeric impurity resembles structurally with the drug substance and elutes very closely (0.2-0.3 min. difference) with the drug substance in reverse phase HPLC and creates a great deal of separation problem.

In the second stage of the 286'process, a slurry of hydroxylamine hydrochloride and sodium hydroxide/sodium carbonate was used. To this mixture ethyl-2-ethoxymethyleneacetoacetic acid ester was added. When the condensation with ethylacetoacetate with ethyl orthoformate followed by cyclization with hydroxylamine hydrochloride are carried out sequentially without isolation or purification of the intermediate, ethyl-2-ethoxymethyleneacetoacetic acid ester, the process suffers from the production of high level of isomeric impurities. This in turn leads to side-reactions during subsequent acid formation and from there to the end product, 5-methylisoxazole-4-carboxylic-(4-trifluoromethyl)-anilide.

In the third stage of the '286 process, the conversion of ethyl-5-methylisoxazole-4-carboxylate to its corresponding acid was carried out by hydrolyzing with a mixture of glacial acetic acid and concentrated hydrochloric acid in the ratio of 2:1.

Thus the condensation/cyclization and subsequent reaction conditions employed according to the '286 process produced by-products and impurities necessitating an additional chemical step, distillation, to restore the purity of the cyclized ester and thereby the end product. The process suffers in two aspects i) lowering the yield of 5-methylisoxazole-4-carboxylic acid resulting in the yield to the level of 37% and ii) generate an impurity, constitutional isomer, to the level of 10.4% which is very difficult to purify from the drug substance even after distillation in the second step of the reaction or repeated crystallization of the acid in the third step of the reaction. This isomeric impurity is carried forward to the finished crystallized product. Only 1% to 2% lowering of the total impurity has been achieved even after carbon treatment and repeated crystallization.

### EXAMPLE 2

Preparation of Ethyl Ethoxymethyleneacetoacetic Ester

A three-necked RB flask fitted with condenser, nitrogen gas inlet and a dropping funnel, was charged with ethylacetoacetate 90.0 g (0.69 mole), triethylorthoformate 152.55 g (0.84 mole) and acetic anhydride 175.5 g (1.72 mole) and the mixture was heated at 100°-110° C. (oil bath temperature) for 2 hours and 30 minutes under nitrogen blanket. The progress of the reaction was monitored by TLC (n-Hexane:Ethylacetate=7:3) to make sure about the completion of the reaction. The reaction can be stopped when TLC shows approximately less than 5% ethylacetoacetate. After the reaction the mixture was cooled to 50° C. and the liquid in the flask fitted with distillation set was heated again under vaccum at 85°-90° C. for distillation of non-reactive liquid. About 125 mL distillate was collected in 40-45 minutes and when no further distillate was observed, the heating was stopped and the reddish brown syrupy liquid was cooled down to room temperature. Weight of the reddish brown syrup was 185 g (0.99 mole). This crude can go directly to the next step reaction without purification.

### EXAMPLE 3

Preparation of Ethyl-5-Methylisoxazole-4-Carboxylate

Into a three-necked RB flask was charged with 185 g (0.99 mole) of ethyl ethoxymethyleneacetoacetic ester and 89.7 g (1.09 mole) of sodium acetate dissolving in minimum amount of water. To this mixture was added 300 mL ethanol. The mixture was cooled to about −5° C. by acetone-salt-ice mixture. To this cooled reddish brown syrupy liquid (which became solidified initially but became liquid on addition of hydroxylamine sulfate solution) was added dropwise a clear cooled (temperature 3° C. to 4° C.) solution of 89.8 g (0.54 mole) hydroxylamine sulfate dissolving in minimum amount of water with vigorous stirring. Addition was made through a dropping funnel for a period of at least one hour with vigorous stirring while the temperature was maintained minimum 0° C. inside the flask. After addition the stirring was continued at this temperature for another 30 minutes. After this the mixture was allowed to cool to room temperature with stirring. The mixture was then again refluxed for 30 minutes (oil bath temperature 85°-90° C.). After the reaction the solution was brought to room temperature and the supernatant was decanted to remove the salt. Ethanol was removed from the mixture by rotary evaporator (water bath temp. 60° C.) under reduced pressure. The aqueous part was extracted with dichloromethane (200 mL×3). The dichloromethane extract was washed with cold saturated brine water (100 mL×2) and dried over anhydrous sodium sulfate. Removal of dichloromethane in rotary evaporator under reduced pressure resulted in orange liquid (130 g, 85% crude yield).

### EXAMPLE 4

Preparation of 5-Methylisoxazole-4-Carboxylic Acid

A two-necked flask fitted with mechanical stirrer and a horizontal condenser for distillation was charged with 40.0 g of crude Ethyl-5-methylisoxazole-4-carboxylate and 44 g of 60% sulfuric acid and the mixture was heated to 85° C. with continuous distillation of ethanol from the reaction product. After four hours of heating at 85° C., TLC showed the complete disappearance of the upper spot of ester. The mixture was allowed to cool in the refrigerator and the solid acid was filtered (16.5 g) Filtrate kept at room temperature for second crop. Acid was crystallized in 60 mL 2% acetic acid-Toluene to obtain about 99.9% pure acid (9.5 g). Mother liquor of the final crystallization was kept for second crop. Crystallization was accomplished by: The crude acid was taken in 2% acetic acid-toluene mixture and heated for 30 minutes. Brown oil was separated at the bottom of the flask. The clear organic phase was neatly transferred and kept for crystallization.

### EXAMPLE 5

Preparation of 5-Methylisoxazole-4-Carbonyl Chloride

5.2 g (0.04 mole) of crystallized 5-methylisoxazole-4-carboxylic acid, freshly distilled thionyl chloride (4.5 mL, 7.3 g, 0.061 mole) and 50 mL anhydrous toluene were mixed together and refluxed for 2 hours and 30 minutes under nitrogen atmosphere. The light brown color solution was distilled at 85°-90° C. and about 8-10 mL distillate was collected resulting in a light brown color liquid.

### EXAMPLE 6

Preparation of 5-Methylisoxazole-4-Carboxylic-(4-trifluoromethyl)-Anilide

The 5-methylisoxazole-4-carbonyl chloride prepared in Example 4 was charged in a dry three-necked round bottom flask fitted with mechanical stirrer under nitrogen atmosphere with 50 mL dry toluene and cooled to 0° C. To this vigorously stirring mixture was added a mixture of para TFMA (6.28 g, 0.039 mole, on the basis of 95% yield of acid chloride) and triethylamine (3.95 g, 0.039 mole) dropwise through a dropping funnel maintaining the temperature between 0° C. to 4° C. inside the flask and nitrogen atmosphere. After addition the reaction mixture was allowed to stir at room temperature overnight. Solid was filtered and washed with toluene. Organic phase was washed with (6N) HCl, water (until neutral to litmus), dried over anhydrous sodium sulfate and evaporated under reduced pressure resulting in a cream color solid. The cream color solid (4.9 g, yield from acid 52%) obtained was crystallized in toluene resulting in a white crystalline solid. Only 6% loss of the solid was observed in the final crystallization of Leflunomide drug substance. Potency of the solid in HPLC is 99.8%.

### EXAMPLE 7

Table I is a comparison of prior art processes and the process of the invention.

TABLE I

Temp.

| Basic Reagent Used | | Salt of | | During | | % Isomeric |
|---|---|---|---|---|---|---|
| for Cyclization | | Hydroxylamine | Addition Technique | Addition | Distillation | Impurity (HPLC) |
| The ′786 | Strong | NH₂OH.HCl | Na₂CO₃ or | Room | Distillation | CRUDE |
| Process | Base: | | NaOH + aqueous | temp. | can not | 10.4% |
| The ′286 | Na₂CO₃ or | | NH₂OH.HCl, to it | | remove the | DISTILLED |
| Process | NaOH or | | added ethyl | | impurity as | 5.1% |
| | any alkali | | ethoxymethylenea | | this is | |
| | metal | | cetoacetic ester | | isomeric in | |
| | hydroxide | | | | nature. | |
| Process of | Mild Base: | (NH₂OH)₂. | Reverse addition: | About | No need of | CRUDE |
| the | Sodium | H₂S0₄ | Ehyl | −5 ° C. | distillation. | 2.5% |
| Invention | Acetate | | ethoxymethylene | | The reaction | |
| | | | acetoaceticester + | | mixture can | |
| | | | ethanolic | | go directly to | |
| | | | aqueous NaOAc, | | the next step. | |
| | | | to it added pre-cooled | | | |
| | | | (NH₂OH)₂.H₂SO₄ | | | |
| | | | solution dropwise | | | |

The results in Table I show that a reverse addition technique has been employed successfully, i.e., previously cooled (3° C. to 4° C.) hydroxylamine sulfate solution is added dropwise to the reaction mixture at the temperature of −5° C., providing an additional regioselectivity towards ethoxymethylene carbon to bring down the percentage of isomeric impurity to the level of 24%.

### EXAMPLE 8

Table II is a comparison of prior art processes and the process of the invention.

TABLE II

| | Hydrolysis of Ethyl-5-methylisoxazole-4-carboxylate and | Solvent Used for | % Isomeric |
|---|---|---|---|
| | its Isomeric Impurity | Crystallization | Impurity |
| The Process of the Invention | 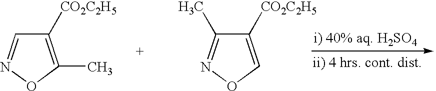 | i) Wash the solid with conc. CH₃COOH | i) 1.5% |
| | 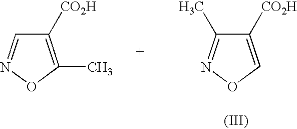 | ii) Crystallization from 2% CH₃COOH- Toluene | ii) 0.1% |
| The ‘786 Process | 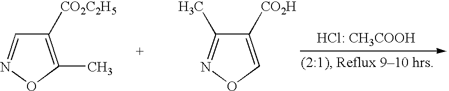 | Crystallization from toluene | ˜8% |
| The ‘286 Process | 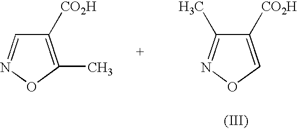 | | |

The results in Table II show that hydrolysis of Ethyl-5-methylisoxazole-4-carboxylate has been accomplished by 60% aqueous H₂SO₄ instead of using a mixture of acetic acid:HCl (2:1) as used in the '786 Process and the '286 Process. Yield is much higher in 60% aqueous H₂SO₄ hydrolysis compare to hydrolysis in the CH₃COOH:HCl (2:1) mixture. Reaction time is also reduced to 3 hours 30 minutes from 9 hours. Prolonged exposure of the ethyl-5-methylisoxazole-4-carboxylate in refluxing condition in acidic medium is prone to generate more by-products. This possibility has been reduced drastically by choosing 60% aqueous H₂SO₄ as a hydrolyzing solvent and continuous distillation process to remove ethanol at the temperature range 80°-88° C.

The results in Table II also show that a novel crystallizing solvent (2% acetic acid—toluene) has been developed to reduce the impurity level from 2.2% (this percentage comes right after the synthesis ) to 0.1% of 5-methylisoxazole-4-carboxylic acid.

TABLE III
PROCESS CONTROL OF BY-PRODUCT IN THE SYNTHESIS OF LEFLUNOMIDE DRUG
SUBSTANCE

| | | Temp. | Addition and | | | % By- |
|---|---|---|---|---|---|---|
| | Mole Ratio:Acid | During | Stirring | | Crystallization | Product |
| | Chloride:TFMA | Addition | Condition | Work-Up | Technique | (CATA) |
| Process | 1:1 | **0 ° C. | TFMA mixed | Organic phase is | 3% CH₃COOH- | 10 ppm |
| I | *1:1.5 | | with | washed with 6 N | toluene as a | *90 ppm |
| | | | triethylamine; | HCl, then washed | crystallization | |
| | | | added dropwise | with water, dried, | solvent | |
| | | | to acid chloride | evaporated under | | |
| | | | in toluene, with | reduced pressure | | |
| | | | mixing*** | | | |
| Process | 1:1 | **0 ° C. | TFMA mixed | Organic phase is | Toluene as a | 6 ppm |
| II | | | with | mixed with 6 N | crystallizing | |
| | | | triethylamine | HCl and stirred | solvent | |
| | | | added dropwise | for 20-30 | | |
| | | | to acid chloride | minutes, washed | | |
| | | | in toluene with | with water, dried, | | |
| | | | vigorous | evaporated under | | |
| | | | mixing*** | reduced pressure | | |

**Much lower temperature suppresses the reactivity and accumulation of TFMA in the medium, which can lead to the formation of by-product CATA. Higher temperature increases entropy favorable for the abstraction of proton from 3 position of the isoxazole ring.
***A slow addition of TFMA and triethylamine by vigorous stirring provides immediate dispersion, thus limiting the localized basified zone, which generate the by-product CATA.

The results in Table III show that the process of the present invention reduces the formation of the by-product (CATA) to a ppm level. A reverse addition technique of TFMA mixed with triethylamine to the acid chloride of 5-methylisoxazole-4-carboxylic acid at low temperature with proper stirring eliminate the possibility of generation of CATA. Proton at 3-position of isoxazole ring can easily be abstracted in basic medium and/or high temperature.

The results in Table III also show that prevention of basic environment has been accomplished in the process of the present invention by slow reverse addition of TFMA and triethylamine to the acid chloride solution in dilute condition and efficient stirring for immediate dispersion of basic amine before accumulation. Vigorous stirring by mechanical stirrer to limit the localized basified zone and lowering of temperature of the reaction medium to decrease the entropy of the medium, thus control the content of the by-product in the drug substance. Work up and crystallization techniques have also attributed the quality product of the final drug substance. Thus, the process of the invention produces Leflunomide that contains CATA in less than about 100 ppm, and most preferably, less than about 12 ppm.

The process of the invention is especially advantageous for preparing 5-methylisoxazole-4-carboxylic-(4′-trifluoromethyl)-anilide, since the process: (1) eliminates or reduces the formation of the by-product CATA, generally as low as 0.0006%; (2) eliminates or reduces the formation of isomeric impurity ethyl-3-methyisoxazole-4-carboxylate and its corresponding acid as low as 0.1%, (3) produces a high quality of 5-methylisoxazole-4-carboxylic-(4′-trifluoromethyl)-anilide generally having 99.8-100% HPLC potency; and (4) does not require distillation of the isoxazole ester.

While the invention has been described with particular reference to certain embodiments thereof, it will be understood that changes and modifications may be made by those of ordinary skill within the scope and spirit of the following claims:

What is claimed is:

1. A process for preparing 5-methylisoxazole-4-carboxylic-(4′-trifluoromethyl)-anilide comprising:
(a) reacting ethylacetoacetate, triethylorthoformate, and acetic anhydride at a temperature of from about 75° C. to about 150° C., to form ethyl ethoxymethyleneacetoacetic ester;
(b) combining the ethyl ethoxymethyleneacetoacetic ester with sodium acetate or a salt of trifluoroacetic acid in the presence of hydroxylamine sulfate at a temperature of from about −20° C. to 10° C., to form ethyl-5-methylisoxazole-4-carboxylate;
(c) reacting the ethyl-5-methylisoxazole-4-carboxylate with a strong acid to form 5-methylisoxazole-4-carboxylic acid;
(d) reacting the 5-methylisoxazole-4-carboxylic acid with thionyl chloride to form 5-methylisoxazole-4-carbonyl chloride; and
(e) reacting the 5-methylisoxazole-4-carbonyl chloride with trifluoromethyl aniline and an amine base at a temperature of from about 0° C. to about 50° C. to form 5-methylisoxazole-4-carboxylic-(4′-trifluoromethyl)-anilide.

2. A process for preparing 5-methylisoxazole-4-carboxylic-(4′-trifluoromethyl)-anilide comprising:
(a) reacting ethylacetoacetate, triethylorthoformate, and acetic anhydride at a temperature of from about 90° C. to about 120° C., to form ethyl ethoxymethyleneacetoacetic ester;
(b) combining the ethyl ethoxymethyleneacetoacetic ester with sodium acetate or a salt of trifluoroacetic acid in the presence of hydroxylamine sulfate at a temperature of from about −20° C. to 0° C., to form ethyl-5-methylisoxazole-4-carboxylate;
(c) reacting the ethyl-5-methylisoxazole-4-carboxylate with a strong acid to form 5-methylisoxazole-4-carboxylic acid;
(d) reacting the 5-methylisoxazole-4-carboxylic acid with thionyl chloride to form 5-methylisoxazole-4-carbonyl chloride; and
(e) reacting the 5-methylisoxazole-4-carbonyl chloride with trifluoromethyl aniline and an amine base at a temperature of from about 0° C. to about 20° C. to form 5-methylisoxazole-4-carboxylic-(4′-trifluoromethyl)-anilide.

3. A process for preparing 5-methylisoxazole-4-carboxylic-(4′-trifluoromethyl)-anilide comprising:
(a) reacting ethylacetoacetate, triethylorthoformate, and acetic anhydride at a temperature of from about 75° C. to about 150° C., to form a mixture comprising ethyl ethoxymethyleneacetoacetic ester and a non-reactive component;
(a′) separating the non-reactive component from the mixture formed in Step (a) to yield ethyl ethoxymethyleneacetoacetic ester;
(b) combining the ethyl ethoxymethyleneacetoacetic ester formed in Step (a′) with sodium acetate or a salt of trifluoroacetic acid in the presence of hydroxylamine sulfate at a temperature of from about −20° C. to 10° C., to form crude ethyl-5-methylisoxazole-4-carboxylate;
(b′) purifying the crude ethyl-5-methylisoxazole-4-carboxylate to form ethyl-5-methylisoxazole-4-carboxylate;
(c) reacting the ethyl-5-methylisoxazole-4-carboxylate formed in Step (b′) with a strong acid to form 5-methylisoxazole-4-carboxylic acid;
(c′) crystallizing the 5-methylisoxazole-4-carboxylic acid to form crystallized 5-methylisoxazole-4-carboxylic acid;
(d) reacting the crystallized 5-methylisoxazole-4-carboxylic acid with thionyl chloride to form 5-methylisoxazole-4-carbonyl chloride; and
(e) reacting the 5-methylisoxazole-4-carbonyl chloride with trifluoromethyl aniline and an amine base at a temperature of from about 0° C. to about 50° C. to form 5-methylisoxazole-4-carboxylic-(4′-trifluoromethyl)-anilide.

4. The process according to claim 1 wherein Step (b) is carried out in the presence of a solvent.

5. The process according to claim 4 wherein the solvent is an alcohol.

6. The process according to claim 5 wherein the alcohol is ethanol.

7. The process according to claim 2 wherein Step (a) is conducted at a temperature of from about 100° C. to about 110° C.

8. The process according to claim 2 wherein Step (b) is conducted at a temperature of from about −10° C. to 0° C.

9. The process according to claim 8 wherein the temperature is about −5° C.

10. The process according to claim 2 wherein Step (e) is conducted at a temperature of from about 5° C. to 15° C.

11. The process according to claim 1 wherein the strong acid is selected from the group consisting of sulfuric acid, hydrochloric acid, phosphoric acid, and combinations thereof.

12. The process according to claim 3 wherein Step (c′) is carried out in the presence of a solvent.

13. The process according to claim 12 wherein the solvent is selected from the group consisting of toluene, acetic acid, ethyl acetate, acetonitrile, 1,2-dichloroethane, 1,1-diethoxypropane, 1,1-diethoxymethane, isopropyl ether, dimethyl acetamide, chloroform, methylene chloride, ethylene chloride, carbon tetrachloride, chlorobenzene, and combinations thereof.

14. The process according to claim 13 wherein the solvent is a toluene and acetic acid mixture.

15. The process according to claim 1 wherein Step (d) is carried out in the presence of a solvent.

16. The process according to claim 15 wherein the solvent is selected from the group consisting of toluene, ethyl acetate, acetonitrile, 1,2-dichloroethane, dimethyl acetamide, chloroform, methylene chloride, ethylene chloride, carbon tetrachloride, chlorobenzene, and combinations thereof.

17. The process according to claim 16 wherein the solvent is toluene.

18. The process according to claim 1 wherein the amine base is selected from the group consisting of triethylamine; N,N-diisopropylethylamine; N,N′-diisopropylethylenediamine; and combinations thereof.

19. The process according to claim 18 wherein the amine base is triethylamine.

20. The process according to claim 1 wherein the 5-methylisoxazole-4-carboxylic-(4′-trifluoromethyl)-anilide is substantially free of 2-cyanoacetoacetic-1-(4′-trifluoromethyl)-anilide.

21. The process according to claim 20 wherein the 5-methylisoxazole4-carboxylic-(4′-trifluoromethyl)-anilide contains less than 100 ppm of 2-cyanoacetoacetic-1-(4′-trifluoromethyl)-anilide.

22. The process according to claim 21 wherein the 5-methylisoxazole-4-carboxylic-(4′-trifluoromethyl)-anilide contains less than 12 ppm of 2-cyanoacetoacetic-1-(4′-trifluoromethyl)-anilide.

23. 5-Methylisoxazole-4-carboxylic-(4′-trifluoromethyl)-anilide prepared by a process comprising:
(a) reacting ethylacetoacetate, triethylorthoformate, and acetic anhydride at a temperature of from about 75° C. to about 150° C., to form ethyl ethoxymethyleneacetoacetic ester;
(b) combining the ethyl ethoxymethyleneacetoacetic ester with sodium acetate or a salt of trifluoroacetic acid in the presence of hydroxylamine sulfate at a temperature of from about −20° C. to 10° C., to form ethyl-5-methylisoxazole-4-carboxylate;
(c) reacting the ethyl-5-methylisoxazole-4-carboxylate with a strong acid to form 5-methylisoxazole-4-carboxylic acid;
(d) reacting the 5-methylisoxazole-4-carboxylic acid with thionyl chloride to form 5-methylisoxazole-4-carbonyl chloride; and
(e) reacting the 5-methylisoxazole-4-carbonyl chloride with trifluoromethyl aniline and an amine base at a temperature of from about 0° C. to about 50° C. to form 5-methylisoxazole-4-carboxylic-(4′-trifluoromethyl)-anilide.

24. 5-Methylisoxazole-4-carboxylic-(4′-trifluoromethyl)-anilide prepared by a process comprising:
(a) reacting ethylacetoacetate, triethylorthoformate, and acetic anhydride at a temperature of from about 75° C. to about 150° C., to form a mixture comprising ethyl ethoxymethyleneacetoacetic ester and a non-reactive component;
(a′) separating the non-reactive component from the mixture formed in Step (a) to yield ethyl ethoxymethyleneacetoacetic ester;
(b) combining the ethyl ethoxymethyleneacetoacetic ester formed in Step (a′) with sodium acetate or a salt of trifluoroacetic acid in the presence of hydroxylamine sulfate at a temperature of from about −20° C. to 10° C., to form crude ethyl-5-methylisoxazole4-carboxylate;
(b′) purifying the crude ethyl-5-methylisoxazole-4-carboxylate to form ethyl-5-methylisoxazole-4-carboxylate;
(c) reacting the ethyl-5-methylisoxazole-4-carboxylate formed in Step (b′) with a strong acid to form 5-methylisoxazole-4-carboxylic acid;
(c′) crystallizing the 5-methylisoxazole-4-carboxylic acid to form crystallized 5-methylisoxazole-4-carboxylic acid;
(d) reacting the crystallized 5-methylisoxazole-4-carboxylic acid with thionyl chloride to form 5-methylisoxazole-4-carbonyl chloride; and
(e) reacting the 5-methylisoxazole-4-carbonyl chloride with trifluoromethyl aniline and an amine base at a temperature of from about 0° C. to about 50° C. to form 5-methylisoxazole-4-carboxylic-(4′-trifluoromethyl)-anilide.
